(19) United States
(12) Patent Application Publication
(10) Pub. No.: US 2021/0350910 A1
(43) Pub. Date: Nov. 11, 2021

(54) SYSTEM AND METHOD FOR SUPPORTING HEALTHCARE COST AND QUALITY MANAGEMENT

(71) Applicants: Shahram Shawn Dastmalchi, San Ramon, CA (US); Charles A. Schuetz, Piedmont, CA (US)

(72) Inventors: Shahram Shawn Dastmalchi, San Ramon, CA (US); Charles A. Schuetz, Piedmont, CA (US)

(21) Appl. No.: 17/200,738

(22) Filed: Mar. 12, 2021

Related U.S. Application Data
(63) Continuation of application No. 15/900,670, filed on Feb. 20, 2018, now abandoned.
(60) Provisional application No. 62/460,704, filed on Feb. 17, 2017.

(51) Int. Cl. G16H 40/20; G06Q 10/04; G06Q 10/10; G06Q 40/00; G06Q 10/06

(52) U.S. Cl. CPC G16H 40/20; G06Q 10/04; G06Q 10/067; G06Q 40/12; G06Q 10/1097

(57) ABSTRACT

Roughly described, historical healthcare records are linked by patient and searched for instances of an episode of healthcare interaction, anchored by a predefined anchor event. Meaningful input and output variables applicable to the episode are identified. Machine learned models are developed which predict the effect that input process variables have on the output variables, and these models are written to a process change exploration data store. Then, through a GUI, a user interactively explores various schedules for changing physical healthcare processes, and the system visually forecasts resulting changes in total cost of an EHI, Planned process changes are implemented and the system visually tracks actual progress both of input process variable implementation and output variable changes. The user can use this information to modify implementation schedules for input process changes. The system can also predict total cost of a specific ongoing EHI long before actual financial claims data are available.

### CROSS-REFERENCE TO RELATED APPLICATIONS

This Continuation Application claims the benefit and priority of U.S. patent application Ser. No. 15/900,670, of the same title, filed Feb. 20, 2018 (Attorney Docket No. ARK1 1001-2), pending, which claims the benefit and priority of U.S. Provisional Patent Application No. 62/460,704, entitled “System and Method for Supporting Health Care Cost Management,” filed Feb. 17, 2017 (Attorney Docket No. ARK1 1001-1), expired, both applications are incorporated herein in their entirety by this reference.

### STATEMENT REGARDING PRIOR DISCLOSURES BY THE INVENTOR OR A JOINT INVENTOR UNDER 37 C.F.R. 1.77(B)(6)

Services implementing some of the concepts described herein were described by the inventors in publicly available literature no earlier than 19 Feb. 2017. Additionally, a website by the inventors promoting services implementing some of the concepts described herein was publicly available no earlier than the same date. Some of such services are not discernable from the literature or the web site posting alone.

### BACKGROUND

The present disclosure relates to computer-aided planning, simulation and modelling of healthcare delivery systems for improving clinical outcomes at the specific patient level and for decreasing total cost of care at the population level.

Healthcare delivery systems that are responsible for health outcomes and cost of care must understand and anticipate their patients' total medical cost. This is not an easy task, since existing systems and methods for obtaining and tracking the cost of care do not typically provide a complete picture of all services rendered to their patients until months later, when financial claims data become available. Even then, the data sets available in healthcare administration derived from electronic health record systems, medical imaging systems, electronic (patient) communication systems, billing systems, and so on are so massive and complex that they cannot be analyzed with traditional data approaches such as SQL databases.

Further, understanding the cost of resources and services is one thing, while understanding the root causes that drive cost is an entirely different challenge. And the relationship between clinical actions and outcomes or cost can be probabilistic rather than deterministic, therefore requiring a complex analysis to account for any cross-interaction between the actions and their estimated impact. Still further, retrospectively knowing the cost of resources rendered to a patient does not automatically provide an understanding of how to reduce cost across a population without negatively impacting quality. In fact, attempts at lowering utilization may reduce short-term cost by decreasing patient's use of cost-effective preventative services, which in turn may degrade patient health outcome and increase costs long term.

### SUMMARY

Though the journey towards value-based care and contracting is often challenging, there are specific actions that healthcare organizations can take to set the stage for financial and operational success. Performance focus areas typically include reducing process variation, improving patient experience, and improving outcomes that are direct contributors to both claims cost and provider operational expense. Performance improvement can be daunting, however, because financial and administrative teams often face several levels of uncertainty: which process-level improvement opportunities should be focused on; how much performance improvement is realistic in a given period; what evidence is needed to convince clinicians to change behavior; what process-level improvements are necessary to achieve the desired outcome; and what is an expected return on investment?

Aspects of the technology disclosed herein apply predictive analytics to answer these questions, and to bring financial confidence to health systems and their partner health plans. In one aspect actionable insights are discovered by analysis of not only financial claims, but also by connecting such claims to the clinical, operational and patient-reported healthcare data that describes features of an episode of healthcare interaction (EHI) that resulted in such claims —even though the time frames in which such data become available vary widely. The combined data are used to understand how specific healthcare process features affect the outcome or the total cost of care, at a highly granular level.

In one embodiment, roughly described, historical healthcare records are collected and linked by patient identifier. They are then searched to find instances of a predefined type of EHI, anchored by a predefined anchor event. All the data for each such episode is then reduced to a set of meaningful “features” applicable to the episode, some of which may be considered input variables and others output variables. Some of the input variables are input process variables, which may be subject to change for future outcome improvement. From the episode of healthcare interaction data, a machine learned set of models is developed which predict the effect that each of the input process variables have on each of a plurality of the output variables, and these models (along with other information) are written to a process change exploration data store. The system then uses the data store in a variety of ways to effect and track process improvement. For example, using a graphical user interface, a user can specify a schedule by which one or more selected input process variables will be changed. The system will then forecast the resulting change in one or more of the output variables and plot it on the graphical user interface. The user can adjust the targets interactively, visually observe the effect on performance, iteratively until a plan is ready. A number of other GUI-based visuals are also provided based on the historical data to assist the user in the exploration process. Once the process changes called for in the plan begin to be implemented, because the system continues to ingest raw data periodically, the actual progress of new episodes of healthcare interaction of the same type are identified and tracked. The system can visually track actual input process variable implementation against the implementation schedule, and can visually forecast the effect on forecast output variable changes of any deviations of the actual input process variable changes from those targeted in the plan. The user can use this information to modify the implementation schedule for one or more of the input process variables, and/or redouble efforts to implement a process change that is lagging targets. In addition, because the process change exploration data store also contains unit cost data for each of the model output variables, the system can also predict the total cost of a specific individual episode of healthcare interaction of the predefined type long before actual financial claims data are available.

At the administrator level, roughly described, the models can also be used to make frequent (e.g. daily) outcome-based cost estimates, which are made available to hospital administrators and clinical personnel via the graphical user interface. In various embodiments, the system can provide administrators with timely running estimates of average episode cost, high-impact sources of variation, and all patient episodes that are being actively managed by the risk-bearing entity (e.g. a hospital). The system enables administrators to see the impact of sources of variation expressed in terms of dollars of average episode cost, thereby simplifying interpretation and prioritization of process improvements.

The above summary is provided in order to provide a basic understanding of some aspects of the invention. This summary is not intended to identify key or critical elements of the invention or to delineate the scope of the invention. Its sole purpose is to present some concepts in a simplified form as a prelude to the more detailed description that is presented later.

### BRIEF DESCRIPTION OF THE DRAWINGS

The invention will be described with respect to specific embodiments thereof, and reference will be made to the drawings, in which:

FIG. 1 illustrates process and data flow for ingesting, analyzing and reducing source data for preparing an EHI patient database.

FIG. 2 illustrates process and data flow for processing data from the EHI patient database of FIG. 1 and preparing a process change exploration data store.

FIG. 3 illustrates process and data flow for exploring and otherwise using the process change exploration data store of FIG. 2 for healthcare process improvement and other purposes.

FIG. 4 illustrates an Episode of Healthcare Interaction.

FIG. 5 illustrates a logical flow for executing the model in the process change exploration data store of FIG. 3.

FIG. 6 illustrates an example GUI form in which a user can enter target values.

FIGS. 7, 8, 12 and 13 are example plots generated by the GUI tool of FIG. 3.

FIG. 9 is a benchmarking visualization generated by the GUI tool of FIG. 3.

FIGS. 10 and 11 illustrate example opportunity forecaster plots generated by the GUI tool of FIG. 3.

FIG. 14 illustrates a distributed computer system that can be used for construction, updating, and management of episode of healthcare interaction data structures.

FIG. 15 illustrates components of the Data Engine in the architecture of FIG. 14.

FIG. 16 illustrates a computer system architecture that can be used to implement computer components in the system described herein.

### DESCRIPTION

The following description is presented to enable any person skilled in the art to make and use the invention, and is provided in the context of a particular application and its requirements. Various modifications to the disclosed embodiments will be readily apparent to those skilled in the art, and the general principles defined herein may be applied to other embodiments and applications without departing from the spirit and scope of the present invention. Thus, the present invention is not intended to be limited to the embodiments shown, but is to be accorded the widest scope consistent with the principles and features disclosed herein.

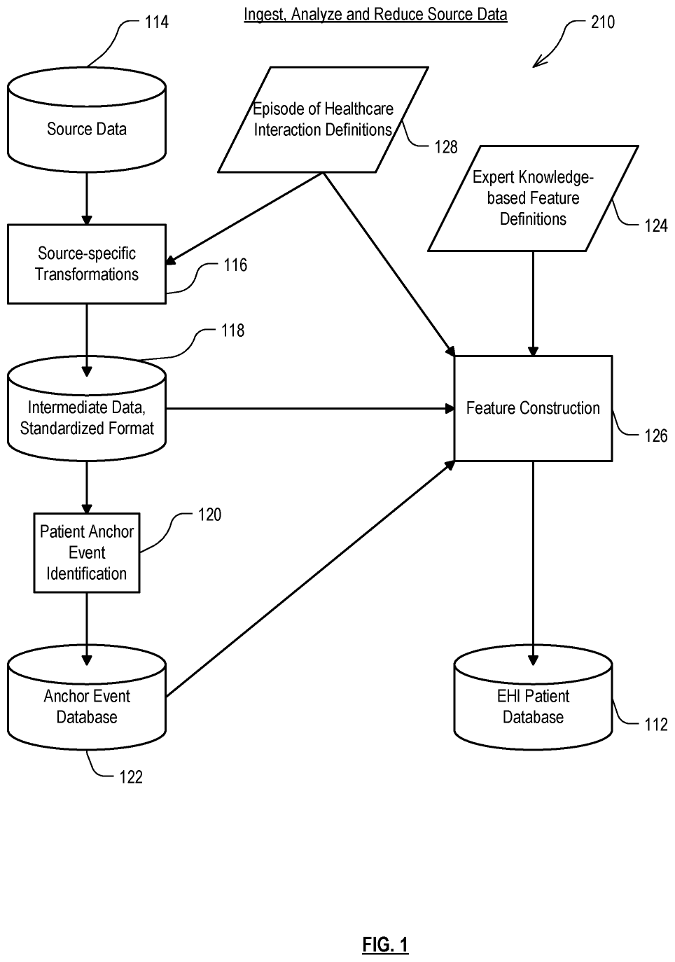

FIGS. 1, 2 and 3 are diagrams illustrating various aspects of the flow and manipulation of data according to an embodiment of the invention. In broad overview, and roughly described, in FIG. 1, historical source data from both patient clinical records and financial claims records are ingested. As used herein, “clinical” records or “clinical” data are medical data about a particular patient, including vitals (such as blood pressure, weight, heart rate), clinical actions and records of same (such as encounter records, procedures, notes, diagnosis codes, referrals, flow sheet data, etc.), laboratory measures, medications, and messages among healthcare personnel. They do not include financial claims records or data. The records of interest are at patient-level, rather than at the level of any aggregation of patients. The records are analyzed to identify “episodes of healthcare interaction” (EHI) in the data, for some episode of healthcare interaction type that is of interest (for example “hip replacement without fracture”). The system writes into an episode of healthcare interaction patient data database 112 information about each identified episode, including the total cost of the particular episode (obtained from the ingested financial claims data), and the presence or absence or quantity of various predefined “features” in each of the episodes of healthcare interaction. The features include input process features (such as whether or not the patient walked 300 steps prior to discharge), as well as output features (such as the patient's in-hospital length-of-stay (LOS)), and are obtained from the ingested patient medical records.

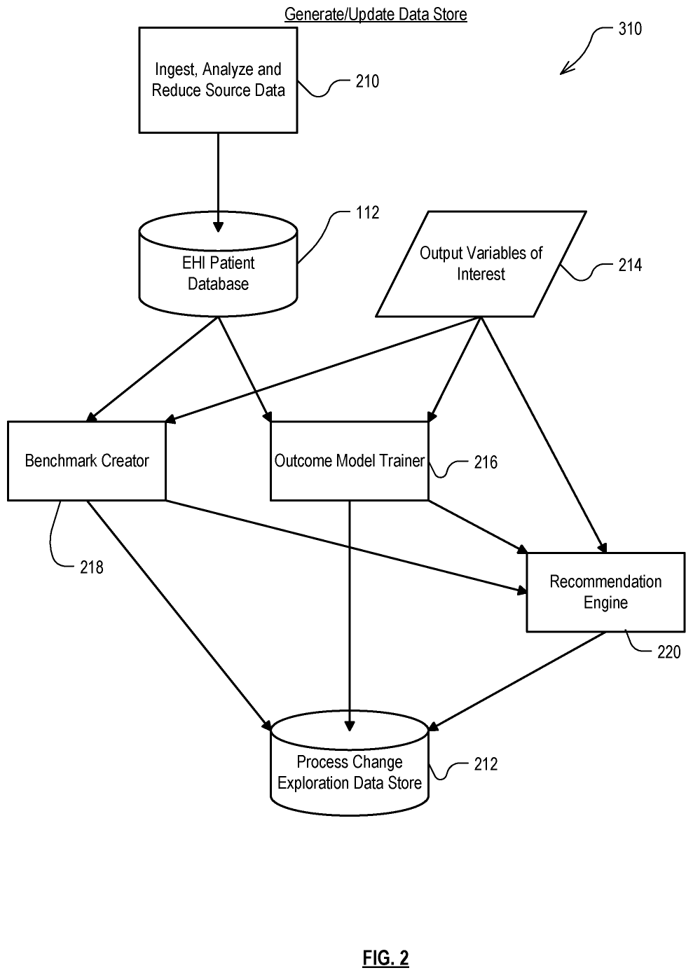

In FIG. 2, the system analyzes the EHI patient database to, among other things, train a model that forecasts the effect that changes in each of a number of the process features will have on one or more of on the output features, or on the total cost (or other aggregation) of an EHI of the predefined type. The “features” of the EHI are now considered input and output variables of the predictive model. The model is written into a process change exploration data store 212.

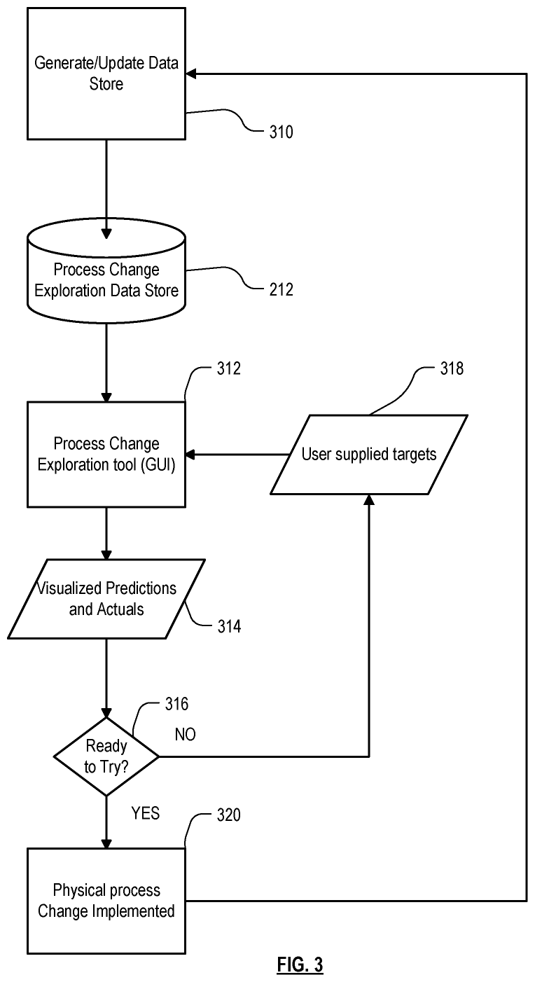

In FIG. 3, the system provides a graphical user interface (GUI) that helps a user in a number of unique ways to identify targeted changes to the healthcare processes, forecast results, and then track progress during implementation and update the forecasts based on actual data.

Referring to FIG. 1, a diversity of data 114 from multiple sources is provided. The source data can include records from hospital or healthcare delivery system electronic health records (EHR's), records of financial claims made to a payer, a cost accounting data warehouse, reported wearable device records, messaging, home monitoring, and so on. Ingestion can be performed with encrypted storage (e.g. portable hard drive), or secure web service (e.g. secure FTP). Some data may be ingested in real-time through web services. Other data may be ingested monthly, quarterly, or even annually as it becomes available. Each ingested record at this point is specific to a single patient, but the system has not yet determined which records belong to which episodes of healthcare interaction. Some of the records might be extremely recent, even created while an EHI is ongoing, whereas other records may not be available until several months after an EHI completes, such as total financial claims data.

Traditional Health Technology platforms (e.g. EHR) have had a transactional focus. These traditional systems are designed to document events that generate revenue for the healthcare provider. These systems typically have not had the ability to generate or maintain an episode of healthcare interaction data structure as is presently described. Traditional systems have also been built on relational database systems, and such database systems can scale poorly. Further, the volume of data ingested in managing a large population (many million patients) where each day numerous patients are entering, progressing through, and exiting episodes of healthcare interaction is substantial. Patient information is most valuable to the healthcare delivery system if it is timely, so the system converting the raw data to episode of healthcare interaction data structures should have high computational throughput and good scalability. In an embodiment, the construction, updating, and management of such episode of healthcare interaction data structure is performed by a distributed computer system such as that shown in FIG. 14.

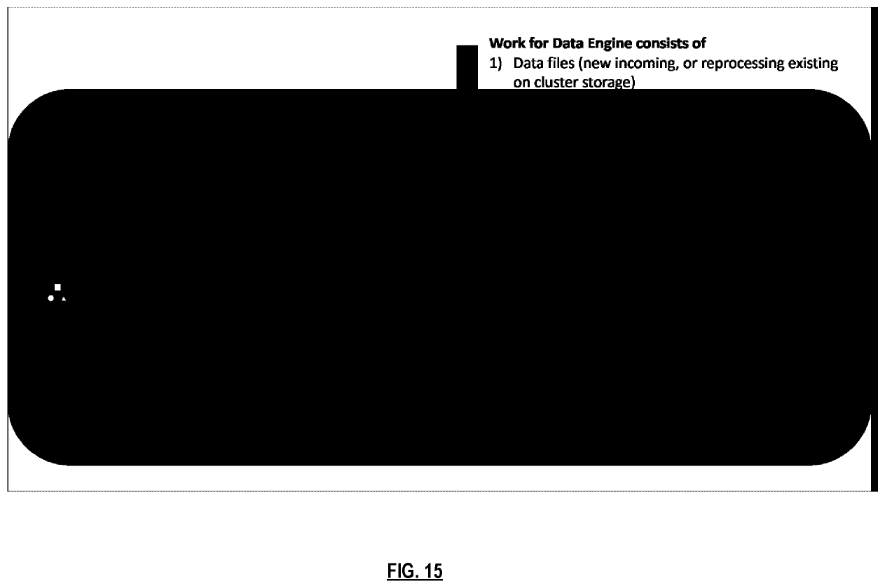

FIG. 15 illustrates components of the Data Engine in the architecture of FIG. 14. The system architecture includes the following components:

1. High-capacity distributed storage system (e.g. Hadoop File System, HDFS) capable of ingesting massive amounts of data cost effectively, using commodity hardware.

2. A web-service responsible for managing the identities of patients, and patient episodes of healthcare interaction.

3. A number of distributed worker computers (that are attached to the distributed storage system) and in communication with the identity management service.

The episode of healthcare interaction management system functions as follows:

1. A given data set (claims data, EHR data, messaging data, etc.) may be broken up into one or more data payloads and stored on the distributed storage system.

2. When a new payload of data is to be consumed, a task (set of instructions and target data) is given to one of the distributed workers.

3. The worker then completes that task, parsing filtering, and transforming the data file, and querying the identity management system as needed to attribute each relevant data element in the file to a particular patient and episode of healthcare interaction.

4. The output from the worker is a set of machine learning features that are each attributable to specific patient and episodes of healthcare interaction.

In box 116, the incoming data records are transformed from source-specific schemas to an internal intermediate standardized schema. A globally unique patient identification number is applied to each individual in the incoming data, which can be used as an index in the subsequent steps. Existing patients in the system also are reconciled against new incoming patients to resolve duplicate patient records.

In order to be able to link data ingested from a variety of different data sets, patients who will enter, or are currently in an episode of healthcare interaction, are identified consistently at a variety of phases during the episode. Embodiments of the system herein can identify patients in some or all of the following phases of an episode of healthcare interaction:

1. Patient may be pre-procedure (e.g. the EHI has not started, but is forecasted or planned to start in the near future).

2. Patient has a scheduled procedure date that would place them in an EHI.

3. Patient had a scheduled procedure date that date was cancelled

4. Patient is admitted to hospital for anchor event procedure.

5. Patient is post-acute, and still within an episode of healthcare interaction. In this category, the patient may be:

a. At a skilled nursing facility (SNF), at an Inpatient Rehabilitation Facility (IRF), or other facility. These facilities may have varying levels of data sharing with the at risk entity.

b. At home, with or without HH (Home Health Care), and with or without non-medical home care.

c. Presenting at an Urgent Care facility.

d. Presenting at an emergency department (ED). The ED may be at the same hospital as the anchor event or a different hospital. If a different hospital, it may be in the same integrated delivery network or a different one.

e. Readmitting to a hospital. The hospital may be the same hospital where the anchor event occurred or a different hospital. If a different hospital, it may be in the same integrated delivery network or a different one.

Box 116 is also where an initial pass is made over each domain of data (table or collection, for example) to identify all patients meeting the criteria for an “episode of healthcare interaction” of a predefined type. As used herein, an episode of healthcare interaction is a sequence of events related to an “anchor event,” or a period of time defined relative to an anchor event. An “anchor event”, as used herein, is a clinical event or procedure or other marker, defined as part of the definition of the EHI, that defines a reference point for an EHI. The anchor event for example could be a surgical procedure (e.g. joint replacement) or a diagnosis (e.g. cancer) which triggers a care protocol (e.g. outpatient chemotherapy), or a clinical marker (e.g. electrophysiology, body temperature, range of motion). The anchor event may define the beginning of an episode of healthcare interaction, or the episode of healthcare interaction may start some time offset before or after the anchor event. The episode of healthcare interaction will then extend for some time after the anchor event or after certain clinical events that follow the anchor event (e.g. 90 days post hospital discharge). In an embodiment, anchor events are limited to events that can be identified by one or more clinical codes (such as DRG, ICD, CPT, etc.) or similar specification. In another embodiment, anchor events can include so-called engineered features, which are rule-based features that can involve more than one record. For example, one embodiment may define an anchor event as a diagnosis of a specified condition which is followed within 10 days by a specified medical procedure. In another embodiment, the definition of an anchor event for a subject episode of healthcare interaction type identifies a particular kind of a clinical event, but excludes such events if similar clinical events occurred before or after it. In one embodiment, a clinical event immediately following an anchor event will be considered part of the episode of healthcare interaction defined by the first event, rather than a second episode of healthcare interaction.

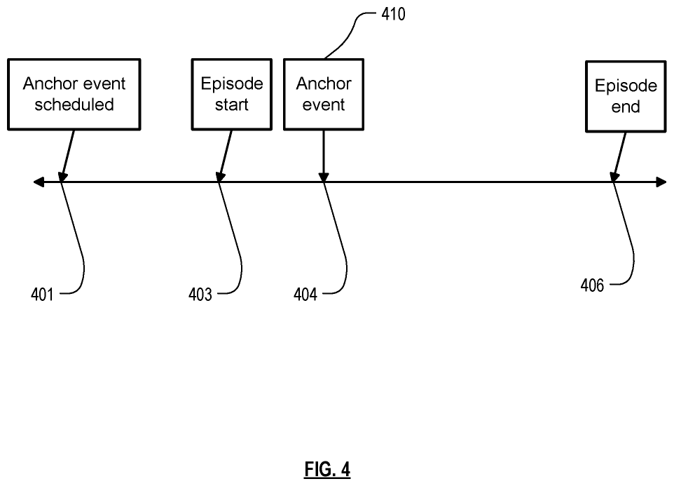

FIG. 4 illustrates an Episode of Healthcare Interaction as the term is used herein, and how it relates to an anchor event 410 in one embodiment. The timeline of FIG. 4 begins with the scheduling 401 of the anchor event. The anchor event for various EHIs is not always pre-scheduled, but in the example of FIG. 4 it was. The EHI itself is defined as beginning at some time 403 prior to the anchor event, and is defined as ending at some time 406 after the anchor event. There may be important events prior to the episode start, such as pre-surgical interventions or patient education. The period 401-404 represents a pre-anchor phase, and the period 404-406 represents a post-anchor phase.

As used herein, an EHI has definite start and end dates, predefined either by dates or rules. The system of FIG. 1 in one embodiment is specific to a single type of EHI that is to be addressed, and the definition for the EHI type is provided in box 128 and referenced in the source-specific transformation box 116. As used herein, an “episode of healthcare interaction” is a single instance of an “episode of healthcare interaction type.” Box 116 makes a preliminary pass over the incoming data to eliminate the records from patients that clearly do not have an EHI of the predefined type. All collected data for each of the remaining patients, cleaned and associated with patient identifiers, are written to an intermediate database 118.

In box 120, the system identifies the presence or absence of qualifying anchor events in the intermediate data. It then collects all of the patient records in the intermediate database, which are dated within the time boundaries of the episode of healthcare interaction anchored by the anchor event, and writes them into the Anchor Event Database. This includes cost information from financial claims data. Note that in one embodiment, it is not necessary that the anchored episode of healthcare interaction has actually concluded; it may still be ongoing.

Individual clinical events in an EHI do not necessarily offer a basis on which to guide process changes that will then impact outcomes or cost of EHIs of the subject type. Therefore, at 124, a collection of engineered features are provided which, when they exist in an EHI, indicate the presence of a higher level concept. Engineered features, as the term is used herein, are variables or values that represent some combination of data elements. For example, a True/False valued engineered feature for whether or not a patient has taken 300 steps during a hospitalization may be constructed from the text of tens of notes recorded by nurses that may be reflected in the patient records collected in the anchor event database 122. The engineered features are typically evidence-based features that have been reported in the medical literature as potentially impacting outcomes or cost of care, and selected by an expert. Another example of an engineered feature would be a particular pain management protocol that has been reported in the medical literature as being potentially impactful. It is not necessary at this point that the engineered features provided in 124 actually have significant impact; the outcome model trainer 216 in FIG. 2 will predict based on the historical records what the impact actually has been for each feature.

In box 126, the system analyzes each of the EHIs represented in the anchor event database 122 and determines presence or absence, or other value, of each of the provided features. The EHIs, each of its features, as well as cost of care data for the EHI, are written into EHI Patient Database 112.

The EHI patient database 112 includes several types of information regarding each included EHI. They include the features from box 124, the total cost of the EHI, and preferably also include metadata about the EHI. The metadata describes the circumstances in which the EHI occurred, such as gender of the patient, marital status, alcohol user, and payer. Many of these are attributes that are not considered subject to modification in order to effect process improvements. The metadata also includes an identifier for a “responsible party” to which an EHI is attributed. The “responsible party” may be a hospital or other facility that managed the care, or it could be a particular physician, a nurse or other caregiver, or any other categorization within which the user desires to improve outcomes. In order to simplify the description herein, most of the discussion refers to responsible parties simply as hospitals.

Many of the features included in the EHI patient database can be divided into input variables and output variables for purposes of the models developed herein. These are all defined initially by 124, but whether to consider a particular variable a “input variable” or an “output variable” depends on the user's goal. For example, if the goal is to reduce length of stay in the hospital (LOS) because each day of stay adds significant cost, then LOS may be defined as an “output variable.” But if a goal is to reduce the incidence of hospital-acquired infections, then LOS may be one of the “input variables.” The term “input variable”, as used herein, is further divided into “input process variables” and other input variables. An ‘input process variable” is an input variable that addresses a healthcare process or group of processes, that a user might addressing for change during process change exploration. Additionally, the term “output variable”, as used herein, includes both “model output variables,” which represent individual output features in the EHIs, and “aggregated output variables,” which aggregate two or more of the model output variables to indicate a combined output value. An example of an aggregated output variable is “EHI total cost”, which as will be seen, involves multiplying the values of model output variables by predetermined unit cost values, and summing the products. Other examples of aggregated output values for various embodiments include clinical performance improvement, patient experience improvement, time savings, and so on. By calculating aggregate output variables, the system can unify all the model outputs to a single number that aggregates the improvements made in all the individual model output variables.

For a particular application of the system, the model output variables are specified in box 214, discussed later with respect to FIG. 2.

EHI patient database 112 contains an episode of healthcare interaction data structure for each qualifying anchor event present in the anchor event database 122. An episode of healthcare interaction data structure is constructed for each combination of a patient and qualifying anchor event. A given patient may have more than one episode of healthcare interaction, and thus be represented in multiple episode of healthcare interaction data structures. As explained further below, those episodes of healthcare interaction data structures can be collected and used to train a model, or if a model exists, can be combined with a model to make a prediction of future health outcomes and cost.

The content of an episode of healthcare interaction data structure can vary by the patient's then-current phase in the given episode of healthcare interaction. Early in the episode, for example, it may contain only patient identity data. For a completed episode, as another example, it may contain patient identity, all clinical events and details captured during the episode, and all financial claims and healthcare resource consumption recorded during the episode. In an embodiment, components of the data structure include immutable attributes of the patient (such as sex, name, address), vitals (such as blood pressure, weight, heart rate), clinical actions and records (such as encounter records, procedures, notes, diagnosis codes, referrals, flow sheet data, etc.), laboratory measures, medications, and messages. The episode of healthcare interaction data structure can include for example the following information:
1. The identity of the patient
   a. Legal name
   b. Internal (gold standard) ID number
   c. A set of source ID numbers for the patient (e.g. one or more patient identification numbers used by the source EHR or other data systems)
   d. Current home address
2. Date stamps that define the beginning and end of the episode of healthcare interaction.
3. Descriptors that define the type of episode (e.g. DRG, ICD-10, etc. codes).
4. A set (or timeline) of events occurring or scheduled to occur during the episode of healthcare interaction. Events that are predicted to occur may be added to the episode. As the patient progresses through the episode, such events will be updated or removed if they don't occur.
5. Events ingested from input data streams may be probabilistically assigned to patients, due to uncertainty in identity or the event occurrence.
6. Confidence estimates describing the uncertainty in attributing data to the given patient episode, uncertainty in occurrence of a predicted future event, and confidence in the final cost of such an event.

The following is a sample data structure used in an embodiment of the system, for maintaining an episode for a patient named Tina Smith, over a number of different phases of an episode of healthcare interaction:

{{“patient_id”: 123, “sex”: “female”, “last_name”: “Smith”,

“first_name”: “Tina”, “dob”: “1930-12-25”, “bundle_id”: 789,
“hospital_id”: 1, “mrn”: 89898, “disease_area”: “lejr”,
“hospital”: “Example Hospital”, “start_date”: “2016-01-16 11:00:00”,
“end_date”: “2016-04-22 14:00:00”, “procedure_physician”: “Dr. Bob”,
“case_summary”: “81 yr. old female, lives alone, diabetes and CKD.”,
“status”: “additional support needed”, “episode_phase”: “post_acute”,
“episode_sub_phase”: “home_w_home_health”, “active_episode”: true,
“recommended_actions”: “Sleep apnea assessment”, “patient_risk”: 1.4,
“modifiable_risk”: 0.5, “risk_reduction_to_date”: 0.0,
“events”: [{“id”: 1, “name”: “Anchor Event Scheduled”,

“start”: “2016-01-01”, “class”: “event”},

{“id”: 2, “name”: “PAT Complete”, “start”: “2016-01-10”,

“class”: “event”},

{“id”: 3, “name”: “Pre-op Home Health”,

“start”: “2016-01-15”, “class”: “event”},

{“id”: 4, “name”: “Inpatient Stay”,
“start”: “2016-01-19 08:00:00”,

“end”: “2016-01-22 14:00:00”},

{“id”: 5, “name”: “Anchor Event”,
“start”: “2016-01-19 11:00:00”,
“class”: “event”},
{“id”: 6, “name”: “Dr. Who Follow-up Visit”,
“start”: “2016-02-16”, “class”: “event”}]}

FIG. 2 illustrates how the system uses the EHI patient database 112 to, among other things, train a model that forecasts the effect that changes in each of a number of the process input variables will have on one or more model output variables.

Referring to FIG. 2, the EHI patient database 112 is developed or updated by box 210, an embodiment of which is illustrated in FIG. 1 and discussed above. In 214 the user specifies the output variables of interest. These are provided, along with the EHI patient database 112, to a benchmark creator 218. The benchmark creator 218 first identifies a set of the patient-level episode of healthcare interaction data structures that capture anchor events occurring during some historical “baseline period”. The EHIs in the EHI patient database 112 whose anchor events occurred during the baseline period are referred to herein as “baseline EHIs”. The system analyzes the baseline EHIs to determine baseline values (such as mean and standard deviation) for each of the included input and output variables. These values are calculated separately for different circumstances represented in the metadata, such as by responsible party. The baseline values are written to the process change exploration data store 212 where they can be used, for example in FIG. 3, to offer the user various benchmarks for potential input process variable targets. They are also used by a recommendation engine 220, as discussed below.

The user specified output variables of interest 214, along with the EHI patient database 112, are also provided to an outcome model trainer 216. The outcome model trainer 216 uses the input variables and corresponding output variables observed in the EHIs, to train one or more predictive models for the output variables of interest using a machine learning algorithm. Different output variables may require differing analytical treatments. Example machine learning algorithms that can be used for various ones of the output variables include Linear Regression, Logistic Regression, and an Artificial Neural Network, among many others. Each episode can contain multiple output variables within it that are modeled.

\In some embodiments, models are trained so as to make the best possible predictions, whereas in other embodiments they are trained so as to best estimate the contribution of process features to the output variables of interest. The former models are best suited to making clinical and financial performance forecasts, whereas the latter models are best suited to guiding quality improvement efforts within a hospital or other responsible party.

The models as trained by the outcome model trainer 216 are written into a process change exploration data store 212. They are represented by a set of values that apply as coefficients to the particular function form used by the outcome model trainer for the particular output variable. For example, if a linear regression algorithm was trained to predict a particular output variable, then the coefficients written into the process change exploration data store 212 for that model may be the weights to be applied to each of the input variables of an EHI in a weighted sum. If a logistic regression algorithm was trained to predict a particular output variable, then the coefficients written into the process change exploration data store 212 for that model may be weights to be applied to the input variables of an EHI in a weighted sum, and then transformed by an inverse logit function. In addition to the model coefficients, the cost distribution of episodes of healthcare interaction of the subject type is estimated from claims data and written into the process change exploration data store 212 as well.

The process change exploration data store 212 in one embodiment contains a model results table in which each pair of an input variable and an output variable is stored as a row. Two examples are:
output variable=LOS,
input variable=Pain Management by Regional+IOExparel+PostOpPCA;
output variable=Admit to SNF (skilled nursing facility),
input variable=Pre-op Patient Education

In an embodiment, different sets of the model coefficients can be stored in the process change exploration data store 122 for different ones of the responsible parties, or divided by other metadata features. The process change exploration data store 122 can also contain other metadata about each EHI, such as sex, marital status, race, smoker, alcohol, etc. Each row includes the model coefficients, and in some embodiments, an indication of the model function form to which the model coefficients apply. For example, function form #1 might be a straight line, which is defined by two coefficients; whereas function form #2 might be a logistic function, which is defined by three coefficients. Each row also includes the unit cost of the output variable on that row. For example, a row in which the output variable is LOS, might indicate a cost per day of LOS.

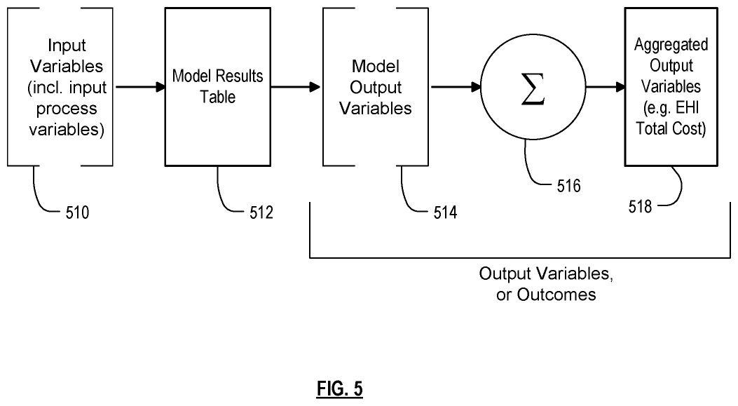

The process change exploration data store 212 thus represents a combined multivariate model for predicting how an output variable will change in response to a change in one or more of input process variables. To predict total cost of a particular EHI, for example, a calculation such as that in FIG. 5 can be used. EHI total cost of care is an example of an aggregated output variable 518 in FIG. 5. A vector 510 containing values for each of the input variables (including input process variables and other input variables), is provided to the model results table 512. For each model output variable, the model results table applies the coefficients to function form for that output variable, and evaluates the resulting function using the provided input variable values. The model results output is a vector 514 of values predicted for each of the output variables. These results are aggregated by an aggregator 516, which yields the value for the aggregated output variable 518. For EHI total cost of care, the aggregation 516 involves multiplying each of the model output variables by a respective predetermined unit cost, and all of the products are summed to obtain the predicted EHI total cost. A user can determine how a change in one or more of the input process variables will affect EHI total cost by varying the relevant input variable values in the input vector 510 accordingly, and observing the resulting cost 518. If the user is interested in the effect of an input process variable change on a particular model output variable, rather than on an aggregated value, this can be viewed in the model output variables vector 514 and the aggregation 516 can be omitted. The methodology of FIG. 5 is sometimes referred to herein as “executing the model”.

In order to assist a user to focus on the most likely impactful process features to try changing with respect to the particular responsible party of interest (sometimes referred to herein as the “subject” responsible party), the system of FIG. 2 also includes a recommendation engine 220. The recommendation engine 220 receives the identification of the output variables of interest 214, the models from the outcome model trainer 216, as well as the benchmarks from benchmark creator 218. The recommendation engine 220 determines, for each of at least some of the input process variables, an “opportunity value” that maximally improves the value of one or more of the output variables, but which is determined by the system according to some predefined definition to be “reasonably attainable.” For example, one of the benchmarks might indicate a mean and standard deviation by which a particular one of the input variables varies over all the responsible parties represented in the EHI patient database 112. The system might be configured to consider two standard deviations above or below the mean to be “reasonably attainable”. Or the system might be configured to consider the minimum and maximum mean values of the input variable over all the represented responsible parties to be “reasonably attainable” values. Other ways can be used in other embodiments for selecting “reasonably attainable” targets. Note that as used herein, a “determination” by the system that a particular value is “reasonably attainable” need not actually be correct; only that a determination has been made.

The recommendation engine 220 then executes the model developed by the outcome model trainer 216 using each of the baseline and most favorable “reasonably attainable” target values for each of the input variables in the input vector 510. The recommendation engine first executes each of the models developed by the model trainer using the baseline input variable values, resulting in a baseline estimate of the output variable. Then, for each input variable of interest, the model is executes at the most favorable “reasonably attainable” target value. (The term “favorable” is used herein to indicate the direction of change that results in improvement of an outcome.) The difference between the model outputs at the “reasonably attainable” favorable target value and baseline value is stored in the process change exploration data store as the benefit of reaching that “reasonably attainable” favorable target value. Thus the process change exploration data store 212 will contain a table which indicates a favorable “reasonably attainable” target for each of the input variables and an indication of the predicted resulting performance improvement for each of the output variables of interest for each of the input variables that is modified to the “reasonably attainable” target.

FIG. 3 illustrates how the system uses the process change exploration data store 212 to, among other things, assist a user to plan a program to improve desired outcomes, in a manner that is realistically attainable. Referring to FIG. 3, the process change exploration data store 212 is developed or updated by box 310, an embodiment of which is illustrated in FIGS. 1 and 2 and discussed above. The data store 212 may for example be in private storage (e.g. a database), or in one embodiment it may by a distributed ledger (e.g. blockchain). An advantage to storing it in a highly secure distributed ledger is that each at-risk entity can have its own copy of the data, including benchmarking data from other entities, and can work with it independently and without having to request permission from other entities. The data store 212 holds model coefficients, user supplied targets, benchmarks, and so on.

In box 312 the process change exploration data store 212 is provided to a process change exploration tool, which provides to the user a graphical user interface (GUI) that offers a wide variety of interactive visually-based services to help the user plan a program for improving one or more subject output variables (including model output variables and aggregated output variables). The GUI may be implemented locally on the same computer that accesses the process change exploration data store 212, or preferably it may be web-based. In an embodiment, the GUI also shows near-real-time progress both for implementation of scheduled changes to input process variables, and for output variables of interest. For example, in a planning stage, the GUI shows, among other things, forecasts indicating how one or more output variables will change over time during a performance period in which process variables are changing in accordance with user-specified targets. Various additional tools are provided on the GUI to help the user set reasonably attainable targets for the input process variables. The user can alter the targets for one or more of the input process variables, as well as their implementation schedule, in order to optimize an implementation plan. Then, during an implementation period in which process changes are actually effected with respect to the responsible party (e.g. hospital), the GUI can plot actual input process variable change implementation against the targets, and actual output variable changes against the forecast, among other things. Box 314 represents the visualizations presented by the GUI.

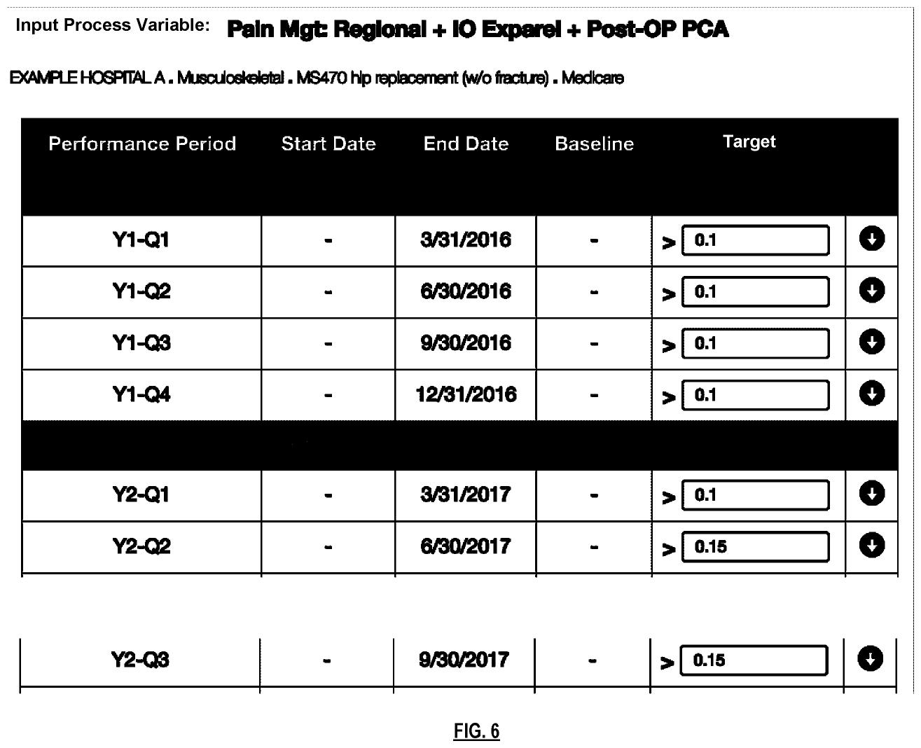

In box 316, the user decides whether the implementation plan is ready to try. If not, then in box 318, the user can alter the user-supplied target values for one or more of the input process variables. Applicant recognizes that it may not be feasible for a healthcare facility to implement a process change suddenly, and that a gradual implementation often is more achievable. In box 318 the user specifies an implementation schedule for one or more of the input process variables, which indicates target values for the input process variable at each of a plurality of times during a performance period. FIG. 6 illustrates an example GUI form in which the user enters target values, gradually increasing each quarter, for an input process variable representing use of “Regional+IO Exparel+Post-OP PCA” as the pain management protocol in specified circumstances. The circumstances are in set forth at the top of the form: Hospital A, category=Musculoskeletal, procedure=hip replacement (w/o fracture), and payer=Medicare. These circumstances are also part of the metadata fields in the process change exploration data store 212. Scheduled implementation is a preferred, but not required, aspect of the invention. In another embodiment, the user may provide a single target value for an input process variable and the system can still use the models in the process change exploration data store 212 to predict resulting values for specified output variables. In yet another embodiment the user can use a form such as that in FIG. 6, but specify the ultimate target value for the input process variable on Day 1. The system can later be used in the implementation period to track the actual extent by which the target value for the input value is achieved over time, as well as the extent to which one or more output variables are improved over time.

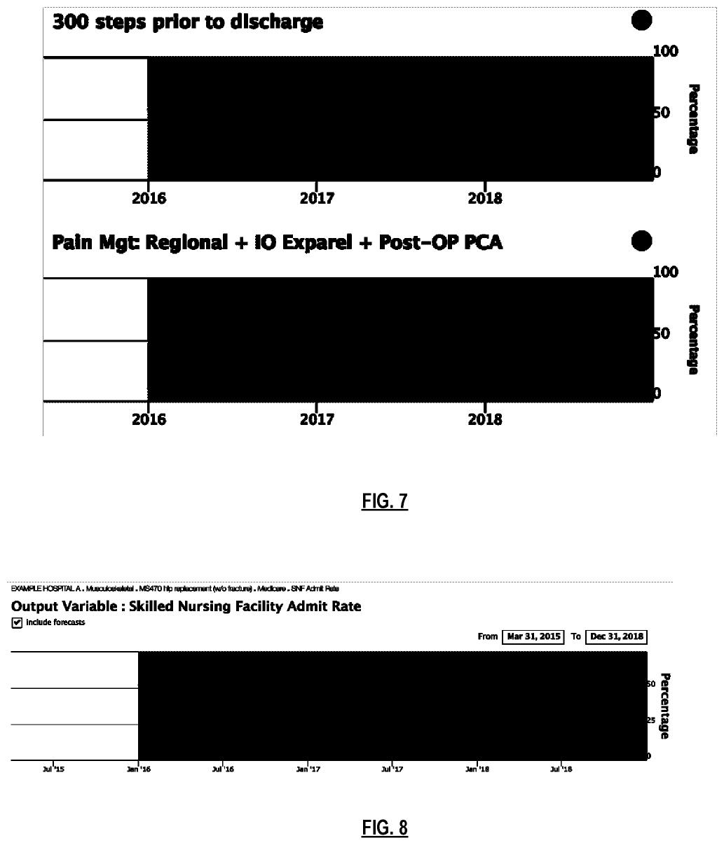

After the user modifies the target values, the process change exploration tool updates the forecasted results and displays them in box 314. The user can interactively and iteratively explore many variations for targets before finally settling on a plan to try implementing. FIG. 7 is an example plot generated by the GUI tool 312 illustrating the user-supplied schedule for implementing two process changes: an increase in the occurrence of process feature “300 steps prior to discharge”, from about 60% to about 80%, and increase in the occurrence of the “Regional+IO Exparel+Post-OP PCA” pain management protocol from about 10% to about 20%. FIG. 8 is a plot generated by the GUI tool 312 illustrating a forecast for model output variable “SNF Admit Rate”, expected if the planned implementation is achieved according to the schedule. This plot is generated by executing the models in the process change exploration data store 212, and forecasts a reduction from about 55% to about 35% in SNF Admit Rate.

The plots of FIGS. 7, 8 and 12 and 13 described below, include a line indicating the scheduled or forecast values over time. The plot is shaded differently above and below this line, one form of shading to indicate the desired region and the other form of shading to indicate the undesired region. For example, in FIG. 8, the because the goal is to push down the percentage of EHIs that include referral to a skilled nursing facility, the region below the forecast line may be green (to indicate desirable values), and the region above the forecast line may be grey (to indicate undesired values).

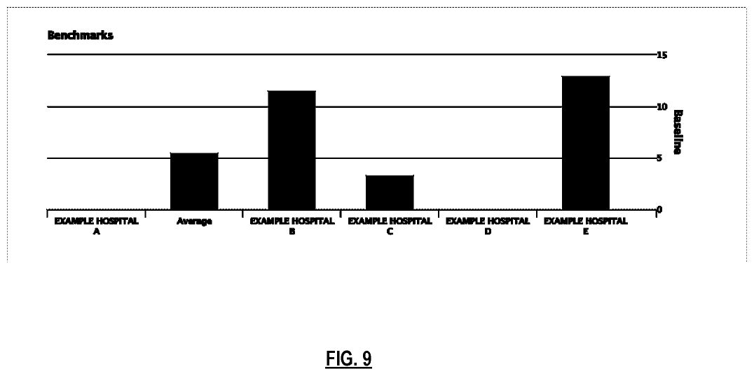

Applicant also recognizes that outcomes may not improve if the target values specified by the user for process changes are not reasonably attainable in the realistic setting of a particular hospital, or other responsible party. In an aspect of the invention, therefore, the process change exploration GUI 314 can display for the user historical mean values for one or more of the input process variables from other responsible parties (e.g. from other hospitals). These values are calculated by the benchmark creator 218 by statistical analysis of the EHI patient database 112 as previously described, and stored in the process change exploration data store 212. FIG. 9 is an example plot which visually shows the mean values of a particular one of the input process variables at four hospitals (B, C, D and E), in addition to the subject hospital, Hospital A. It also shows an average value. These benchmarks help the user, in considering process changes for Hospital A, to understand what values for the particular input process variable may be reasonable attainable in Hospital A.

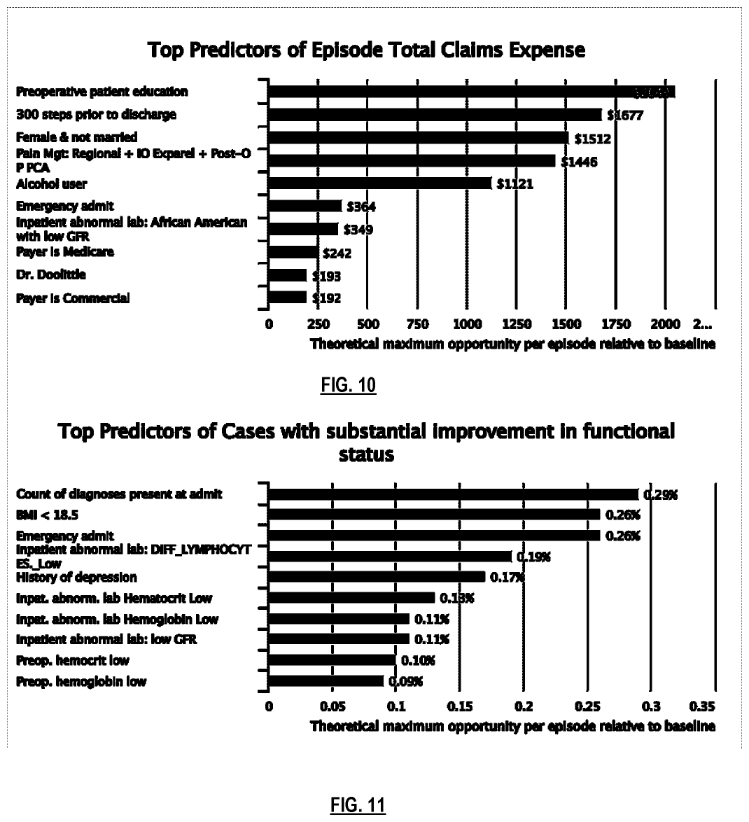

Applicant also recognizes that exploration of process improvements can benefit from guidance about where to start the exploration. Thus in an aspect of the invention, the process change exploration tool 312 visually presents an opportunity forecaster which depicts the maximum improvement that can be expected in response to reasonably attainable changes for each of the top 10 most impactful input process variables. FIG. 10 illustrates an example opportunity forecaster plot for the top 10 input variables most impactful on EHI total cost, and FIG. 11 illustrates an example opportunity forecaster plot for the top 10 input variables most impactful on an outcome variable called “functional status”. It can be seen that for each plot, the input process variable to be changed is listed on the left, and the bar chart indicates how much improvement can be expected if the maximum reasonably attainable change in that input process variable is implemented. The data for these charts is obtained from the process change exploration data store 212, having been written there by the recommendation engine 220 as previously described. These plots help to guide the user to understand which particular input process variables should be considered for change in the user's exploration process.

The example plots in FIGS. 10 and 11 each show the top 10 input variables in decreasing order of predicted impact on the relevant output variable. In other embodiments the order can be different. Also in other embodiments different numbers of opportunities can be shown. Preferably, however, the N top input variables are shown, where 1<N≤10.

Returning to FIG. 3, once the user decides to proceed with an implementation plan (box 316), the implementation period can begin. The implementation period involves causing physical process changes. These are caused to occur for example by an at-risk entity which retained or employed one or more individuals or contracting firms to plan the implementation schedules as described herein. Many different types of physical process changes are possible. The following are a few examples of potential physical process changes for episodes of healthcare interaction that involve elective surgery:
The process change exploration GUI 312 may forecast outcome improvements if an increased number of patients have an additional in-person visit at the hospital or a clinic, prior to the surgery, where their physical and mental health is evaluated and modifiable risk factors are addressed. In the visit, the provider and patient establish a plan for whether or not the patient will go home after surgery. The implementation plan may schedule targets for increasing implementation of this process change by percentages over time. The hospital or clinic may cause the plan to be implemented for example through staff training and workflow modifications required by the hospital. Reminding the patient about the plan can provide additional motivation to the patient, and engage the patients care givers to provide support (transportation, help shopping, help bathing).
The process change exploration GUI 312 may forecast outcome improvements if, instead of each doctor using a personally preferred protocol and set of drugs, an increased number of surgeries of a certain type (such as total knee replacement) use a prescribed standard anesthesia protocol with a prescribed set of drugs (e.g. Regional anesthesia plus intraoperative Exparel plus post-operative patient controlled analgesia). The implementation plan may schedule targets for increasing implementation of this process change by percentages over time. The hospital or clinic may cause the plan to be implemented for example through training meetings with anesthesiologists and specific modifications to the hospital's standard work (as documented in printed processes and protocols against which staff are trained), changes to EHR order sets and the GUIs used by physicians and anesthesiologists during procedures, and drug and supply purchasing.
The process change exploration GUI 312 may forecast lower total cost if all elective surgeries of a given type or type(s) start before 16:00. The implementation plan may schedule targets for increasing implementation of this process change over time taking into account surgeries previously scheduled. The hospital or clinic may cause the plan to be implemented for example through communications with surgeons, medical staff scheduling, and operational process changes requiring scheduling personnel to only schedule such surgeries to start prior to 16:00.
The process change exploration GUI 312 may forecast outcome improvements if an increased number of patients who have a total knee replacement surgery will have a first physical therapy session, within the hospital, on the same day as the surgery. The implementation plan may schedule targets for increasing implementation of this process change over time based on a separate schedule by which staffing and resource changes occur in the hospital. The hospital or clinic may cause the plan to be implemented for example through updating standard work (as documented in printed processes and protocols against which staff are trained), training scheduling personnel, nurses, physical therapists and other; hiring additional physical therapists and appropriate hospital resources so that the hospital has sufficient capacity to deliver timely therapy, and then periodically reviewing rates of same-day physical therapy with the providers that deliver that care to track improvement.

For each process change to be implemented according to the plan, it will be apparent to the skilled reader what physical steps will be taken at the subject hospital or other facility in order to implement them.

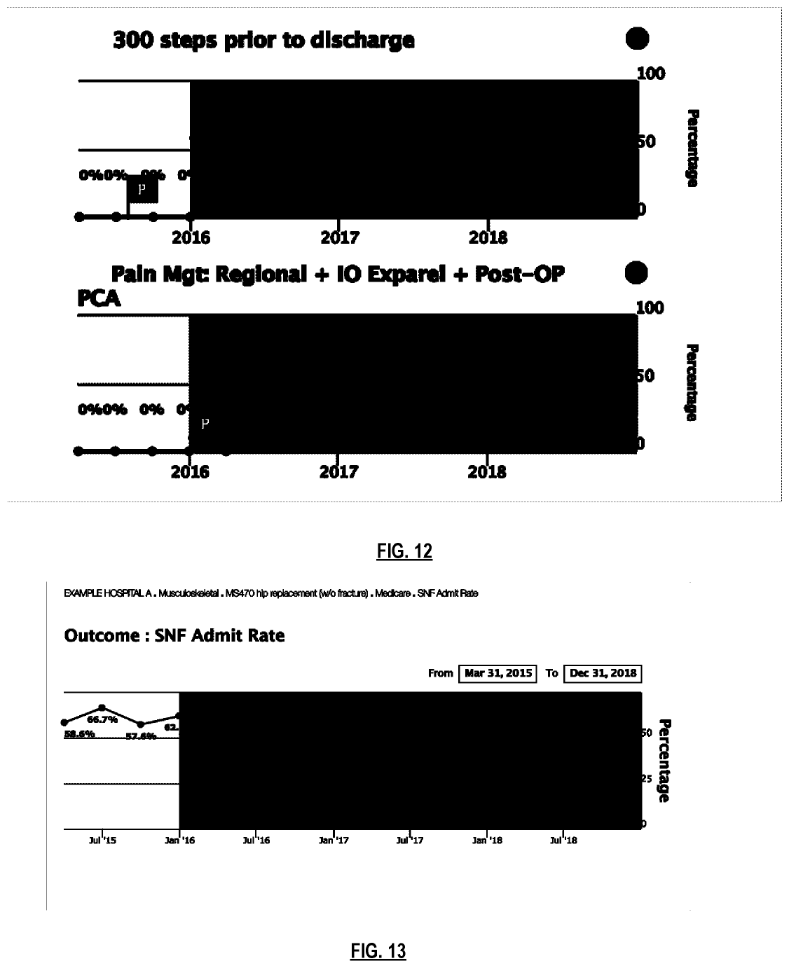

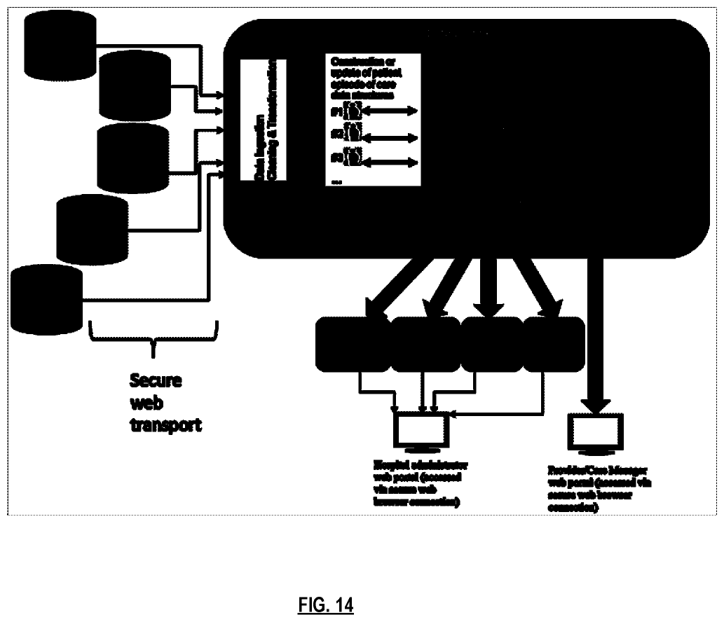

The process in FIG. 1 to create and update the EHI patient database 112 occurs repeatedly in one embodiment, and the updates from various sources can occur at different rates and asynchronously with any particular implementation period. Preferably they continue during the implementation period. Thus as clinical and process data continue to be ingested in FIG. 1, process features of actual EHIs, begun during the implementation period become available in the EHI patient database on an ongoing basis. Thus the system is able to determine the actual extent by which planned process changes are being implemented, and can plot these on the GUI in comparison to the planned schedule. Similarly, the system is able to determine during the implementation period the actual extent to which forecast output variables are being improved. FIG. 12 show plots of the same two input variables depicted in FIG. 7, with an indication of actual implementation compliance over time plotted on the same chart. In the examples of FIG. 12, it can be seen that the input process variable “300 steps prior to discharge” did not meet the initial target of about 60% compliance, but quickly achieved and overcome subsequent targets. Similarly, the input process pain management variable also started out below the scheduled target compliance, but has since increased to match the scheduled targets. FIG. 13 shows the actual progress of improving the output variable “SNF Admit Rate” as a result of the actual compliance level with the scheduled process change targets. It can be seen in FIG. 13 that the output variable “SNF Admit Rate” is improving (decreasing for this variable) at a better rate than forecast.

As a result of these charts, the user can easily see how well the implementation of process improvements is proceeding. In one embodiment, the user or at-risk entity uses the information to modify the implementation schedule for one or more of the input process variables. The process change exploration tool 312 can then be executed for the revised targets, and updated forecasts can be displayed on the GUI. In another embodiment, the user or at-risk entity causes additional physical steps for redoubling efforts to implement a process change that is lagging targets. In addition, because the process change exploration data store also contains unit cost data for each of the model output variables, the system can estimate a total cost to date of an individual, ongoing episode of healthcare interaction, long before actual financial claims data are available.

It is noted that in FIG. 13, the performance of the output variable “SNF Admit Rate” is shown as a percentage of EHIs which exhibit the feature of the specified output variable. In another embodiment, the performance can be shown as an absolute number of EHIs that exhibit the feature. In yet another embodiment the performance can be shown as a measure (such as a percentage) by which the output variable has improved from a baseline value. In yet another embodiment the performance can be shown as a percentage that the actual output variable value bears to the forecast values at each point in time. Many other ways to indicate performance will be apparent to the reader. In general, therefore, the term “performance indication,” as used herein, is intended to cover all ways to indicate such performance.

Using Predictive Models to Provide (Near) Real-Time Health Outcome Risk and Episode Cost Estimates at Both the Patient and Population Level, During the Performance Period, without Claims Data (e.g. Using Clinical Data Streams).

Under value-based care arrangements, the hospital usually holds financial risk for the total cost of care provided to its patients. A system as described herein provides hospitals with previously unavailable foresight into their financial risk and guidance on how to mitigate that risk.

The following analyses are performed on data from the baseline period.

1. The total episode of healthcare interaction cost is broken down into components corresponding to categories of utilization, such as inpatient stay (DRG payment), physician services, medical device, skilled nursing, readmission, etc.

2. The cost variability (e.g. variance) of each cost component is assessed.

3. The cost components with low variation (e.g. DRG payment to the hospital) are estimated as constants, or simple functions (e.g. shallow decision tree, etc.). This constant or simple function is used to estimate the category cost during the performance period.

4. For the cost components with high variation, multivariate predictive models are trained on both the clinical and claims data captured during the baseline period. The models predict clinical and cost outcomes without using claims data.

During the performance period, outcomes and costs are estimated either by outcomes-based cost modeling or by direct estimation, or both, in different embodiments.

For cost modeling using the occurrence of outcomes:

1. For each variable cost category, predictive models are used to estimate the occurrence of health outcomes using EHR (and other non-claims) data features as inputs.

2. At a population level, cost is estimated as the product of the event occurrence rate times the unit cost of the event.

3. At a patient level, cost is estimated as the product of the estimated event occurrence (e.g. 1 or 0) and the unit cost.

For direct estimation:

1. Patient-level EHR and claims data are combined for episodes occurring during the baseline period.

2. A predictive model is trained to estimate claims cost, using only EHR (or other non-claims) data features as inputs.

3. With this model, patient and population-level estimates of cost can be made using only clinical data.

### Details of a Patient-Level Method for Predicting (Near) Real-Time Costs.

The system is used by hospital-based care managers to care for patients. The system has a GUI that provides hospital staff with a patient level view of risk estimates for patients as well as recommendations for optimal care.

In an embodiment for an elective hospital-based procedure (e.g. lower extremity joint replacement (LEJR)), the method begins when the patient is scheduled for the procedure at a hospital or has some other event that makes surgery likely. At this time, the system creates a patient episode of healthcare interaction data structure for the present episode of healthcare interaction.

Following scheduling, the patient is evaluated during a televisit or office-based visit. At this visit, information about the patient's attitude, health, medical history, family support, and living situation is collected and recorded in the EHR. For example, the following may be collected and noted in the patient's episode data structure:

1. Age, diseases, medical history, allergies, smoking, drug and alcohol use, etc.

2. The living situation of the patient, to assess what types of pre- and post-surgical care will be required. Does the patient live with others or alone? Are there family members of friends that can provide post-discharge support. Does the patient sleep on the ground floor of their home, or upstairs, etc.

3. Does the patient have a history of opioid use, anxiety about the procedure etc.

The system estimates health outcome risk and episode cost based on the clinical information collected at the pre-surgical visit, EHR data for the patient, and population-level data about the patient's neighborhood and community. The episode cost is estimated as a composite of the variable and fixed cost components as described above.

If the system estimates that the patient will have a higher than average episode cost, and if that cost is modifiable, then cost reducing services are recommended by the system to the care team and physician (for example via a secure web-based portal, or mobile phone), so that the services can be offered to the patient. The recommendations are made based on model based estimates of which clinical actions will decrease total episode cost most. The cost reducing services correspond to terms in the predictive models used to estimate outcome risk and cost (output variables). For example, if the patient is estimated to have a high episode cost, and lives at home, the system might recommend home health providers visit the home prior to surgery to help the patient prepare their home to receive them after hospital discharge. The system might also recommend physical therapy if the patient is not yet strong enough for surgery. The system might suggest delaying in the surgery, while the patient gains strength or receives therapy, if such a delay is predicted by the model to decrease total episode cost without harming the patient.

On the day prior to the procedure, the system risk scores the patient and re-estimates total episode cost based on all available clinical information. At this point the system might recommend postponing the procedure if the modifiable risk is too high. For patients having an emergency procedure, the present method begins here.

At hospital admission, the patient's risk is re-scored, and episode cost is re-estimated using the suite of predictive models and most recent patient data (taken from the episode of healthcare interaction data structure). At this time, the system recommends an optimal care path for the patient, which could include physician actions, drugs, medical devices and implant selection, counseling, physical therapy, and discharge preparation interventions.

After the surgical procedure, new clinical data becomes available in the EHR. The system risk scores the patient and re-estimates episode cost, this time incorporating detailed information about the surgical procedure (e.g. physician notes and orders, elapsed time in the operating room, blood loss, etc.). The system again makes optimal care path recommendations.

Over the remainder of the hospital stay, the system continues to make updated risk estimates for the patient based on all available clinical data, and alerts the care team in the case that a patient has rising modifiable risk for avoidable cost (for example high risk of inappropriate discharge to a skilled nursing facility).

Upon discharge (when the discharge disposition is known) the system re-estimates the total episode cost, and patients with high risk of modifiable cost can be prioritized by care management and care coordination team members.

The system tracks the patient (via periodic mining of the EHR data) and updates estimates for outcome risk and total episode cost as new information becomes available.

### Details of a Method for Predicting Population Level (Near) Real-Time Costs.

A system as described herein can be used periodically by hospital administrators to minimize the average population-level episode cost, without negatively impacting quality or patient experience metrics. In this aspect, hospital staff periodically review (via a graphical user interface) a variety of metrics provided by the system. The following metrics may be provided in an embodiment of the system:

1. Up-to-date estimates of the average episode of healthcare interaction cost (before claims data becomes available), and the factors that are driving avoidable cost and cost variability.

2. Risk adjusted performance metrics of physicians, nurses, and other clinical actors, e.g. a physician's positive or negative contribution to clinical outcomes and episode cost adjusted for other risk factors. For hospital-based procedures this can be a performance score for how well the physician performs (adjusted for other risk factors) expressed in terms of clinical risk (e.g. is the physician associated with greater or lower than average risk) and the financial impact on average episode price (e.g. dollars). In some embodiments there is also a similar score for outpatient episodes of healthcare interaction, home health provides, non-medical home health personnel, etc.

3. Measures of intervention (care service) effectiveness and impact on average episode of healthcare interaction cost.

Hospital staff also can periodically review the results from the system to prioritize, for example:

1. Physician engagement and education, quality improvement programs, and additional investment.

2. Review predictive modeling results that isolate the true causal variables that predict the occurrence of high variability cost components.

3. Quantifying the financial impact of compliance to quality measures on episode of healthcare interaction cost.

4. Estimating future performance year average episode of healthcare interaction cost by using predictive models combined with performance improvement targets/plans set by the at-risk entity (e.g. hospital).

5. Periodically reconciling prior financial predictions with new batches of financial claims data, updating predictions for the remainder of the performance period. Early in the performance period, claims data will not yet be available for the episodes of healthcare interaction, so episode cost is estimated with predictive models that do not require claims data inputs. Later in the performance period, as claims data becomes available, prior estimates made with predictive models are reconciled against the claims data actual costs. Performance-year-to-date estimates are made by combining claims data (for the episodes for which claims are available) with cost estimates made with predictive models that take only clinical data as inputs.

### Machine Generation of a Concise Textual Summary of a Patient-Level Episode of Healthcare Interaction

Embodiments of the system can automatically generate a concise textual summary of a patient-level episode of healthcare interaction, derived from EHR, claims, and message data. Examples are shown in the “Case Summary” column of the above LEJR drawing. In order to accomplish this, raw data is parsed, and evaluated in terms of importance as a predictor of outcomes and episode cost. Pertinent results are stored in the episode of healthcare interaction data structure. A short textual summary of the patient history, clinical profile, and risk is generated by assembling the most important data elements in a natural language form. The machine generated, concise text is automatically populated in the web-based portal to communicate case details between providers, or/and generate text for notes recorded in other systems.

### Expressing Patient Risk as a Single Number

Embodiments of the system also can quantify patient risk and express it as a single number, referred to herein as an Episode Cost Ratio (ECR). The ECR expresses the cost and clinical risk of each patient as a single number. A patient expected to have a total episode cost equal to the target cost will have an ECR of 1.0. Patients with expected cost greater than the target cost will have an ECR greater than unity, and those with expected cost less than the target cost will have an ECR less than unity. An example of this is shown in the “Patient Risk” column of the above LEJR drawing. The ECR is computed as follows:

1. A predictive model of episode cost is applied to a patient level episode of healthcare interaction data structure, yielding an estimated episode cost for that patient.

ĉ=f(r)

where ĉ is the estimated episode of healthcare interaction cost for a patient, f is the predictive model function, and r is the patient-level episode of healthcare interaction data structure expressed as a vector of risk factors.

2. The estimated cost is normalized by target cost, providing the episode cost ratio.

$$ECR = \frac{\hat{c}}{{c}_{target}}$$

where C_{target} is the target episode cost.

3. In some embodiments, the ECR is adjusted by one or more additive or multiplicative factors, to account for attributes of the healthcare system (rates of missing diagnoses, etc.), seasonality, population attributes, insurance plan attributes, etc.

The ECR can also be computed for a population by replacing a patient level r with one reflecting population averages.

### Certain Particular Features

It can be seen that whereas EHR systems (computer systems) are well suited to billing for care delivered by the at-risk hospital, and being paid retrospectively, they lack the facility to forecast the cost of care, and utilization outside the hospital. At-risk hospitals are financially responsible for the cost of all care delivered during an episode of healthcare interaction. If average total episode exceeds the target cost, then the at-risk entity will be forced to pay the difference retrospectively. Embodiments of the present system address the shortcoming of the EHR, and provides clinical and financial outcome forecasts.

It can be seen also that whereas EHR systems capture clinical and financial data, they do not do so in a form that is amenable to machine processing. Embodiments of the system described herein parse the raw EHR data, composed of codes, free text, images. From the raw data it constructs engineered features, which have well defined values (e.g. Boolean or continuous numerical quantities describing patient history and care delivered). The system then assembles episode of healthcare interaction data structures for each qualifying episode of healthcare interaction. These data structures are amenable to machine processing.

### System Hardware

The following computer hardware drawing is a simplified block diagram of an example computer system 1610 that can be used to implement the high-capacity distributed storage system, the distributed worker computers, the benchmark creator, the outcome model trainer, the recommendation engine, the process change exploration tool 612, and all other computer components in the system described herein. In some embodiments different hardware components of the overall system are implemented using different versions of the example computer system 1610.

Computer system 1610 typically includes a processor subsystem 1614 which communicates with a number of peripheral devices via bus subsystem 1612. These peripheral devices may include a storage subsystem 1624, comprising a memory subsystem 1626 and a file storage subsystem 1628, user interface input devices 1622, user interface output devices 1620, and a network interface subsystem 1616. The input and output devices allow user interaction with computer system 1610. Network interface subsystem 1616 provides an interface to outside networks, including an interface to communication network 1618, and is coupled via communication network 1618 to corresponding interface devices in other computer systems. Communication network 1618 may comprise many interconnected computer systems and communication links. These communication links may be wireline links, optical links, wireless links, or any other mechanisms for communication of information, but typically it is an IP-based communication network. While in one embodiment, communication network 1618 is the Internet, in other embodiments, communication network 1618 may be any suitable computer network.

The physical hardware component of network interfaces are sometimes referred to as network interface cards (NICs), although they need not be in the form of cards: for instance they could be in the form of integrated circuits (ICs) and connectors fitted directly onto a motherboard, or in the form of macrocells fabricated on a single integrated circuit chip with other components of the computer system.

User interface input devices 1622 may include a keyboard, pointing devices such as a mouse, trackball, touchpad, or graphics tablet, a scanner, a touch screen incorporated into the display, audio input devices such as voice recognition systems, microphones, and other types of input devices. In general, use of the term “input device” is intended to include all possible types of devices and ways to input information into computer system 1610 or onto computer network 1618.

User interface output devices 1620 may include a display subsystem, a printer, a fax machine, or non-visual displays such as audio output devices. The display subsystem may include a cathode ray tube (CRT), a flat-panel device such as a liquid crystal display (LCD), a projection device, or some other mechanism for creating a visible image. The display subsystem may also provide non-visual display such as via audio output devices. In general, use of the term “output device” is intended to include all possible types of devices and ways to output information from computer system 1610 to the user or to another machine or computer system.

Storage subsystem 1624 stores the basic programming and data constructs that provide the functionality of certain embodiments of the present invention. For example, the various modules implementing the functionality of certain embodiments of the invention may be stored in storage subsystem 1624. These software modules are generally executed by processor subsystem 1614.

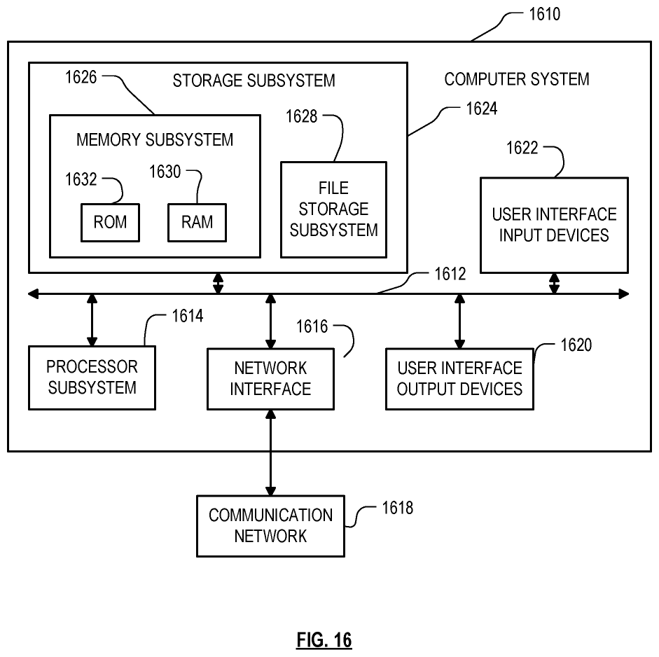

Memory subsystem 1626 typically includes a number of memories including a main random access memory (RAM) 1630 for storage of instructions and data during program execution and a read only memory (ROM) 1632 in which fixed instructions are stored. File storage subsystem 1628 provides persistent storage for program and data files, and may include a hard disk drive, a floppy disk drive along with associated removable media, a CD ROM drive, an optical drive, or removable media cartridges. The databases and modules implementing the functionality of certain embodiments of the invention may have been provided on a computer readable medium such as one or more CD-ROMs, and may be stored by file storage subsystem 1628. For example, the intermediate data store 118, the anchor event database 122, the EHI patient database 112, and the Process Change Exploration Data Store 212 can all be stored in memory subsystem 1626 of one or more computer systems like that of FIG. 16. Alternatively, one or more of such databases can be stored in separate storage that is accessible to the computer system. Additionally, as used herein, no distinction is intended between whether a database is disposed “on” or “in” a computer readable medium. Additionally, as used herein, the term “database” does not necessarily imply any unity of structure. For example, two or more separate databases, when considered together, still constitute a “database” as that term is used herein. Thus in some embodiments of the system herein, two or more of the intermediate data database 118, the anchor event database 122, the EHI patient database 112, and the Process Change Exploration Data Store 212, can be combined into a single structure. Similarly, one or more of such databases in some embodiments can be split into two or more structures that must be accessed separately. Other variations will be apparent.

The host memory 1626 contains, among other things, computer instructions which, when executed by the processor subsystem 1614, cause the computer system to operate or perform functions as described herein. As used herein, processes and software that are said to run in or on “the host” or “the computer”, execute on the processor subsystem 1614 in response to computer instructions and data in the host memory subsystem 1626 including any other local or remote storage for such instructions and data.

Bus subsystem 1612 provides a mechanism for letting the various components and subsystems of computer system 1610 communicate with each other as intended. Although bus subsystem 1612 is shown schematically as a single bus, alternative embodiments of the bus subsystem may use multiple busses.

Computer system 1610 itself can be of varying types including a personal computer, a portable computer, a workstation, a computer terminal, a network computer, a television, a mainframe, a server farm, client/server arrangement, or any other data processing system or user device. Due to the ever-changing nature of computers and networks, the description of computer system 1610 depicted in FIG. 16 is intended only as a specific example for purposes of illustrating the preferred embodiments of the present invention. Many other configurations of computer system 1610 are possible having more or less components than the computer system depicted in FIG. 16.

In an embodiment, software code portions for performing any of the functions described herein can be stored at one location, and then retrieved and transmitted to the location of a computer system that will be executing them. The transmission may take the form of writing the code portions onto a non-transitory computer readable medium and physically delivering the medium to the target computer system, or it may take the form of transmitting the code portions electronically, such as via the Internet, toward the target computer system. As used herein, electronic transmission “toward” a target computer system is complete when the transmission leaves the source properly addressed to the target computer system.

As used herein, a given event or value is “responsive” to a predecessor event or value if the predecessor event or value influenced the given event or value. If there is an intervening processing element, step or time period, the given event or value can still be “responsive” to the predecessor event or value. If the intervening processing element or step combines more than one event or value, the signal output of the processing element or step is considered “responsive” to each of the event or value inputs. If the given event or value is the same as the predecessor event or value, this is merely a degenerate case in which the given event or value is still considered to be “responsive” to the predecessor event or value. “Dependency” of a given event or value upon another event or value is defined similarly.

As used herein, the “identification” of an item of information does not necessarily require the direct specification of that item of information. Information can be “identified” in a field by simply referring to the actual information through one or more layers of indirection, or by identifying one or more items of different information which are together sufficient to determine the actual item of information. In addition, the term “indicate” is used herein to mean the same as “identify”.

The foregoing description of embodiments of the present invention has been provided for the purposes of illustration and description. It is not intended to be exhaustive or to limit the invention to the precise forms disclosed. Obviously, many modifications and variations will be apparent to practitioners skilled in this art. In particular, and without limitation, any and all variations described, suggested or incorporated by reference in the Background section of this patent application are specifically incorporated by reference into the description herein of embodiments of the invention. The embodiments described herein were chosen and described in order to best explain the principles of the invention and its practical application, thereby enabling others skilled in the art to understand the invention for various embodiments and with various modifications as are suited to the particular use contemplated. It is intended that the scope of the invention be defined by the following claims and their equivalents.

What is claimed is:

1. A method for healthcare process improvement, comprising:
providing, accessibly to a computer system, a process change exploration data store which includes a database identifying, for each of a plurality of model output variables of episodes of healthcare interaction of a predefined type, coefficients for a model predicting the effect that each of a plurality of predefined input process variables has on an the respective model output variable;
a computer system receiving from a user a first implementation schedule for a first one of the input process variables, the implementation schedule indicating target values for the first input process variable at each of a plurality of times during a performance period; and
a computer system, in response to the process change exploration data store and the received first implementation schedule, calculating and visually forecasting on a graphical user interface forecast performance over time during the performance period of a subject output variable, the subject output variable being a member of the group consisting of a first one of the model output variables and an aggregation of at least a subset of the model output variables.

2. The method of claim 1, further comprising receiving from the user a modified implementation schedule for the first input process variable; and
a computer system visually forecasting on the graphical user interface performance of the subject output variable over time during the performance period, in response to the process change exploration data store and the modified implementation schedule.

3. The method of claim 1, further comprising receiving from the user a second implementation schedule for a second one of the input process variables; and
a computer system visually forecasting on the graphical user interface performance of the subject output variable over time during the performance period, in response to the process change exploration data store and both the first and second implementation schedules.

4. The method of claim 1, comprising a computer system visually forecasting on the graphical user interface performance of both the first model output variable and a second one of the model output variables over time during the performance period.

5. The method of claim 1, wherein the subject output variable is the first model output variable.

6. The method of claim 1, wherein the subject output variable is the aggregation of model output variables.

7. The method of claim 6, wherein the aggregation of model output variables is a total cost of episodes of healthcare interaction of the predefined type.

8. The method of claim 1, wherein providing a process change exploration data store comprises a computer system developing the process change exploration data store in dependence upon a plurality of historical episodes of healthcare interaction of the predefined type, including in dependence upon both clinical features of each of the episodes and financial claims made with respect to the respective episode.

9. The method of claim 1, wherein the model uses a first function form to predict the effect that the input process variables have on the first model output variable, and uses a second function form different from the first function form to predict the effect that the input process variables have on a second one of the model output variables.

10. The method of claim 1, wherein the model is derived from historical episodes of healthcare interaction attributed to a subject responsible party,
further comprising causing a physical healthcare process of the subject responsible party to change during an implementation period, in dependence upon the first implementation schedule for the first input process variable.

11. The method of claim 10, wherein the subject responsible party is a hospital.

12. The method of claim 10, further comprising determining actual values of the first input process variable during the implementation period.

13. The method of claim 10, further comprising a computer system presenting on the graphical, user interface plots depicting actual values of the first input process variable during the implementation period, in comparison to targets in the first implementation schedule for the first input process variable.

14. The method of claim 10, further comprising, after the presentation by a computer system of plots depicting actual values of the first input process variable during the implementation period:
a computer system receiving from a user a revised implementation schedule for the first input process variable; and
a computer system visually forecasting on the graphical user interface a revised performance forecast for the subject output variable over time during the performance period, in response to the process change exploration data store and the revised implementation schedule.

15. The method of claim 10, further comprising a computer system presenting on the graphical user interface plots depicting actual performance of the subject output variable during the implementation period, in comparison to the forecast performance of the subject output variables as presented in the step of visually forecasting.

16. The method of claim 10, further comprising determining actual values of the first input process variable during the implementation period,
wherein the subject output variable comprises a total cost of episodes of healthcare interaction of the predefined type,
further comprising, during the implementation period, a computer presenting on the graphical user interface in comparison with the performance forecast, an estimated value of the actual total cost of episodes of healthcare interaction of the predefined type, calculated in dependence upon the process change exploration data store and the actual values of the first output variable during the implementation period.

17. The method of claim 10, further comprising:
a computer system updating the model coefficients in the process change exploration data store in dependence upon updated historical data received during the implementation period; and
a computer system presenting an updated visual forecast on the graphical user interface of updated forecast values for the subject output variable over time during the implementation period.

18. The method of claim 1, wherein visually forecasting performance over time of the subject output variable comprises visually depicting values over time of the subject output variable.

19. The method of claim 1, wherein visually forecasting performance over time of the subject output variable comprises visually depicting a measure by which values of the subject output variable improve over time.

20. The method of claim 1, wherein the model coefficients in the process change exploration data store are derived from historical episodes of healthcare interaction attributed to a subject responsible party,
further comprising a computer system determining, from historical episodes of healthcare interaction of the predefined type attributed to a plurality of different responsible parties including the subject responsibility party, statistics specific to each of the responsible parties about historical values for each of a plurality of the predefined input process variables;
a computer system presenting at least a subset of the statistics on the graphical user interface so that they can be referenced by the user in developing the first implementation schedule.

21.-48. (canceled)
